(19) United States
(12) Patent Application Publication
(10) Pub. No.: US 2018/0311445 A1
(43) Pub. Date: Nov. 1, 2018

(54) A POSITION DETERMINATION DEVICE FOR DETERMINING AND REGISTERING AN INJECTION LOCATION

(71) Applicant: Nordic Healthcare Advisory ApS, Ølstykke (DK)

(72) Inventors: David Tobias Bøggild-Damkvist, Ølstykke (DK); Rasmus Thomas Tjalk-Bøggild, Frederiksberg (DK)

(21) Appl. No.: 15/766,231

(22) PCT Filed: Oct. 5, 2016
(86) PCT No.: PCT/EP2016/073756

§ 371 Date: Apr. 5, 2018

(30) Foreign Application Priority Data
Oct. 6, 2015 (DK) PA 201570627

(51) Int. Cl. A61M 5/42; G16H 20/17

(52) U.S. Cl. CPC A61M 5/427; G16H 20/17; A61M 2205/3306; A61M 2205/52; A61M 2205/3561; A61M 2205/3592; A61M 2205/3375

(57) ABSTRACT

A position determination device for determining and registering an injection location of administered fluid drug administration on a user. The position determination device includes a position determination element adapted to determine a current location of the position determination device, and one or more processing units adapted to derive data representing an actual injection location based on a current location determined by the position determination unit.

### FIELD OF THE INVENTION

The invention relates generally to embodiments of a position determination device for and method of determining and registering an injection location of self-administered fluid drug administration on a user. Further the invention relates to calibration of such position determination devices.

### BACKGROUND

A growing number of people have a condition involving fluid drug delivery administration where the user themselves deliver the relevant fluid drug (i.e. self-administration) by injection at an appropriate injection location or site using an injection device or the like.

Preferably, the fluid drug to be delivered is insulin but the invention is applicable to all fluid drug delivery devices where a user administers a drug on at least a somewhat regular basis, e.g. several times a day or less frequent like a number of times in a week, etc., by injection into an appropriate body part.

Other examples of user-administered fluid drugs includes MS therapies with interferons and immune suppressives (e.g. like Avonex, Rebif, Copaxone etc.), different hormone therapies (growth hormone, anti-/hormone followup treatments to breast or prostate cancer, etc.).

In such recurring self-injection regimens it is very important that the user does not inject the drug at the same location every time or too often as this may cause ‘pockets’ of the drug to gather under the skin, cause hard lumps and/or cause extra fat deposits to develop as well as the onset of lipohypertrophy and/or similar skin/tissue damages (that may change the way the liquid drug is absorbed by the body at that location), and so on.

Therefore educating the user in injection techniques is often an important part of such treatment regimens and it is often recommended to change/rotate between injections locations. Furthermore, it may be recommended to inject not closer than 3 or even 1 cm (or some other distance(s)) of a previous injection location; at least within a given timeframe.

To remember and keep track of the injections, users may use certain memory systems and/or simple schematics, but compliance has been shown to usually be lowered over time, in particular for user administered injection schemes.

A typical example of patient education material is found e.g. at: http://www.cc.nih.gov/ccc/patient_education/pepubs/subq.pdf

However, a user may still forget where the last injection was made and/or even if rotating, perhaps some body locations are still used more frequently than others.

Some users may also be young children, which may have a harder time sticking rigorously to a recommended rotation scheme and/or remembering it fully all the time.

There is therefore a need for improved injection location handling and/or management.

It would also be a benefit to provide precise data about actual injection location(s).

### SUMMARY

It is an object to alleviate at least one or more of the above mentioned drawbacks at least to an extent.

Furthermore, it is an object to provide improved injection location handling and/or management.

Additionally, it is an object to enable precise data relating to actual injection location(s).

The invention is defined in claim 1.

Accordingly, in one aspect of the present invention, a position determination device for determining and registering an injection location of administered fluid drug administration on a user, wherein the position determination device comprises
a position determination element adapted to determine a current location of the position determination device, and
one or more processing units adapted to derive data representing an actual injection location based on a current location determined by the position determination unit.

In this way, specific data is obtained of where and when one or more drug injections actually have been made. This enables improved injection location handling and/or management of the drug administration.

In some embodiments, the one or more processing units is/are adapted to derive the data representing an actual injection location based on a current location determined by the position determination unit in response to the user administering the fluid drug using a fluid drug administration device.

In some embodiments, the position determination device is further adapted to be secured to the fluid drug administration device.

In some embodiments, the position determination device has a generally hollow cylindrical shape with an opening or cut-out adapted to receive at least a part of the fluid drug administration device when being secured to this.

In some embodiments, the position determination device comprises a locking mechanism adapted to releasably engage with a mating or corresponding locking mechanism of the liquid drug administration device thereby attaching the position determination device at a predetermined location on and/or predetermined orientation in relation to the drug administration device.

In this way, it is ensured by such a locking mechanism that a distance from the position determination device to the needle (or another point of reference) of the drug administration device is known a priori. This avoids the need for re-calibration every time the position determination device is attached to the drug administration device.

In some further embodiments, the locking mechanism corresponds to a locking mechanism of a cap of the drug administration device.

In some further embodiments, the locking mechanism is further adapted to releasably engage with a mating or corresponding locking mechanism of a cap of the liquid drug administration device thereby enabling the cap to be attached to the position determination device (even when the position determination device is attached to the drug administration device).

In some embodiments, the position determination device further comprises a wireless communications unit adapted to automatically transmit the derived data representing an actual injection location to a separate electric device.

In some embodiments, an actual action of the user activating the fluid drug administration device to administer the fluid drug triggers deriving the data representing an actual injection location.

In some embodiments, a mechanical or remote sensor is attached directly to or attached in connection with an activation button of the fluid drug administration device where the mechanical or remote sensor is adapted to detect movement of the activation button of the fluid drug administration device.

A mechanical sensor may e.g. be physically attached or parallel to the activation button. A remote sensor would measure the distance to the activation button and determine any changes in the activation button position in relation to the position determination device.

In some embodiments, the position determination device further comprises a sound sensor adapted to register one or more distinct sounds caused by a user operating the drug administration device to increase, decrease, and/or release a drug dosage as an actual action of the user and wherein the position determination device is adapted to trigger deriving the data representing an actual injection location in response to registering the one or more distinct sounds.

Such distinct sounds are common among insulin administration devices and other types to allow users with impaired sight to hear when the dosage is respectively increased, decreased, or released.

In some embodiments, the position determination device further comprises
a memory and/or storage comprising data representing at least one previous injection location,
at least one indicator element, adapted to signal to the user
   that the current location of the position determination device, as determined by the position determination element, is at a location where it is acceptable or advisable to perform an injection taking into account at least the data representing at least one previous injection location, and/or
   that the current location of the position determination device, as determined by the position determination element, is at a location where it is not acceptable or advisable to perform an injection taking into account at least the data representing at least one previous injection location.

In some embodiments, the position determination element comprises at least one gyroscope and/or at least one accelerometer.

In some embodiments, the position determination device is further adapted to register an actual amount of administered liquid drug and/or a time-stamp when deriving data representing an actual injection location.

In some embodiments, the position determination device further comprises an image sensor or other sensor element adapted to detect an amount of fluid drug being administered by the fluid drug administration device.

In some embodiments, the position determination device is further adapted to
register a starting position in response to the user moving the position determination device to a pre-determined starting position,
register a location of an anchor position in response to the position determination unit determining the movement from the starting position to the anchor position,
register a location of the actual injection location in response to the position determination unit determining the movement from the anchor position to the injection location.

In some embodiments, the position determination device is further adapted to
recommend the anchor position and/or recommend the location of the actual injection location in response to a rotation schema taking into account one or more previously registered actual injection locations.

In some embodiments, the position determination device is further adapted to
register a starting position when the user has moved the position determination device to a starting position,
if used, registering a region in connection with the starting position defining a region suitable for receiving injections,
registering one or more anchor points, by determining their relative position in relation to the starting position, and for each anchor point registering a region defining a region associated with the given anchor point being suitable for receiving injections.

In some embodiments, the fluid drug is one selected from the group consisting of: insulin, drugs for multiple sclerosis therapies with interferons and immune suppressives (e.g. like Avonex, Rebif, Copaxone etc.), drugs for hormone therapies, drugs for growth hormone therapies, and drugs for anti-/hormone follow-up treatments to breast or prostate cancer.

In some embodiments, the position determination device is integrated with a fluid drug administration device and wherein the derived data representing an actual injection location is made available to at least another electric element of the fluid drug administration device.

In some embodiments, the position determination device is for determining and registering an injection location of self-administered fluid drug administration on a user.

In some embodiments, the position determination device is for determining and registering an injection location of administered fluid drug administration on a user administered by a medical professional.

According to another aspect of the present invention is a use of a position determination device according to any one of claims 1-17 to register one or more locations of actual injection locations.

### DEFINITIONS

All headings and sub-headings are used herein for convenience only and should not be constructed as limiting the invention in any way.

The use of any and all examples, or exemplary language provided herein, is intended merely to better illuminate the invention and does not pose a limitation on the scope of the invention unless otherwise claimed. No language in the specification should be construed as indicating any non-claimed element as essential to the practice of the invention.

This invention includes all modifications and equivalents of the subject matter recited in the claims appended hereto as permitted by applicable law.

### BRIEF DESCRIPTION OF THE DRAWINGS

FIG. 1 illustrates a schematic block diagram of embodiments of a position determination device;

FIGS. 2a-2d schematically illustrate different views of one exemplary embodiment of a position determination device e.g. as illustrated in FIG. 1;

FIG. 3a schematically illustrates a perspective view of the position determination device of FIGS. 2a-2d being secured to a fluid drug administration device;

FIG. 3b schematically illustrates a perspective view of the position determination device and the fluid drug administration device of FIG. 3a before the position determination device is secured to the fluid drug administration device;

FIG. 4 schematically illustrates a flow chart of one embodiment of position determination by a position determination device as shown in FIGS. 1 to 3b and embodiments thereof;

FIG. 5 schematically illustrates a flow chart of one embodiment of a method of suggesting an injection location;

FIG. 6 schematically illustrates a flow chart of one embodiment of a calibration method of a position determination device, e.g. as shown in FIGS. 1 to 3b;

FIG. 7 schematically illustrates a separate electric device displaying a ‘heatmap’ view or similar of earlier injections;

FIG. 8 schematically illustrates an embodiment of a locking mechanism for attaching an embodiment of a position determination device to a drug administration device; and

FIGS. 9a and 9b schematically illustrate an embodiment of the position determination device attached to a drug administration device and by itself.

### DETAILED DESCRIPTION

Various aspects and embodiments of a position determination device for and methods of determining and registering an injection location of administered fluid drug administration on a user and calibration of such position determination devices as disclosed herein will now be described with reference to the figures.

When relative expressions such as “upper” and “lower”, “right” and “left”, “horizontal” and “vertical”, “clockwise” and “counter clockwise” or similar are used in the following terms, these only refer to the appended figures and not to an actual situation of use. The shown figures are schematic representations for which reason the configuration of the different structures as well as their relative dimensions are intended to serve illustrative purposes only.

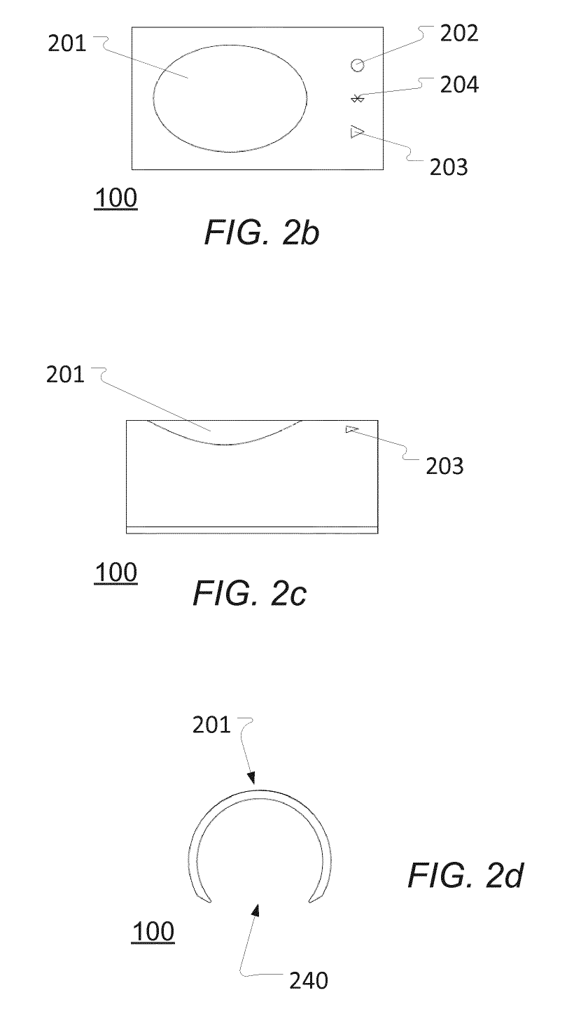

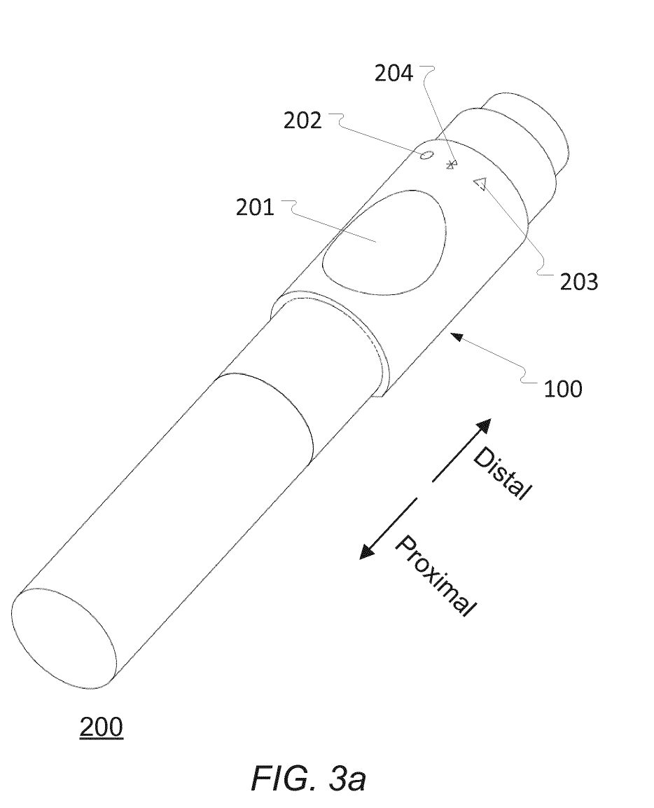

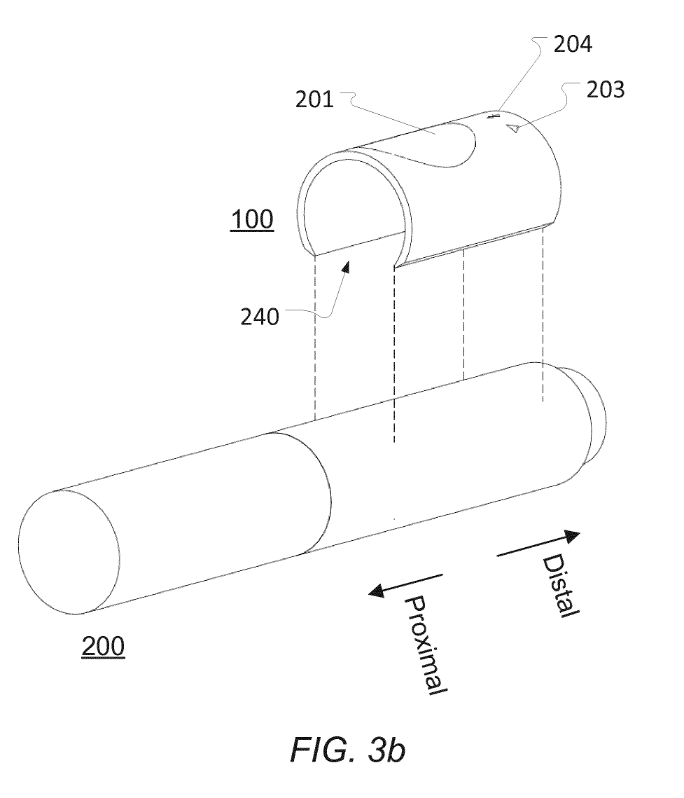

In that context it may be convenient to define that the term “proximal direction” in the appended figures is meant to refer to the direction of the position determination device (and its associated or integrated fluid drug administration device) that normally, during use, would point towards an injection element of the fluid drug administration device , as depicted e.g. in FIGS. 2a and 3a-3b, whereas the term “distal direction” is meant to refer to a direction parallel and opposite to the proximal direction.

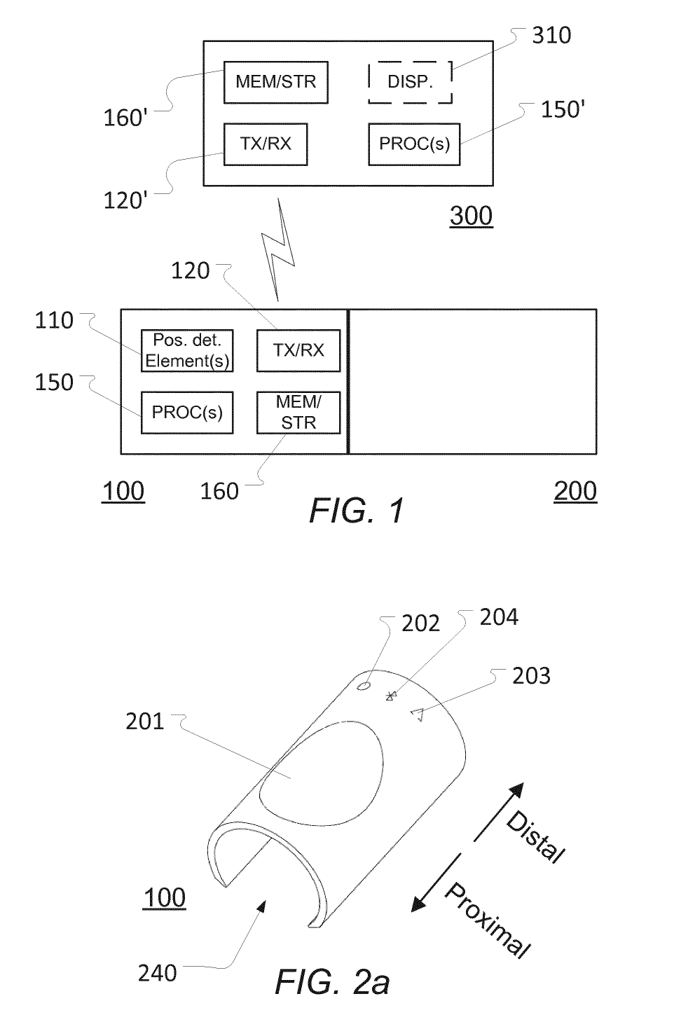

FIG. 1 illustrates a schematic block diagram of embodiments of a position determination device.

Shown is one embodiment of a position determination device 100 and a fluid drug administration device 200. The position determination device 100 may e.g. be integrated with the fluid drug administration device 200 or alternatively be attached or secured to the fluid drug administration device 200, e.g. as shown and explained further in connection with FIGS. 3a-3b and 8-9.

The fluid drug administration device 200 may be of any suitable type that is used by a user or a medical professional to administer a fluid drug at an appropriate injection location on a given user. The fluid drug administration device 200 may e.g. be an insulin administration device such as an insulin pen, insulin doser, insulin pump, etc. Alternatively, the fluid drug administration device may e.g. be a syringe for delivery of interferons, immune suppressives, or hormone therapies.

The position determination device 100 comprises one or more position determination elements 110 and in this particular and other embodiments, one or more processing units 150, at least one digital memory and/or storage 160, and at least one communications unit 120, preferably one or more standardised wireless communications units e.g. like Wi-Fi, Bluetooth, NFC, etc. communications units.

If the position determination device 100 is integrated with the fluid drug administration device 200 they may share one or more elements, such as the processing unit(s), the digital memory/storage, and/or the communication unit(s).

Further shown is a separate electric device 300 comprising its own one or more processing units 150′, at least one digital memory and/or storage 160′, at least one communications unit 120′, and optionally a display 310.

Preferably, the position determination device 100 (and/or the fluid drug administration device 200) may wirelessly communicate or exchange data and information with the separate electric device 300.

The separate electric device 300 may e.g. be a mobile communications and/or computation device such as a smartphone, a tablet, a laptop or PC, etc. or a separate dedicated special purpose device, such as a data-logger or the like.

The position determination device 100 is for determining and registering an injection location of (e.g. self-administered) fluid drug administration on a user as will be explained in the following.

According to an aspect of the present invention, the position determination element 110 is adapted to determine, during use, a current location of the position determination device 100 and the one or more processing units 150 is/are adapted to derive, during use, data representing an actual injection location based on a current location determined by the position determination unit 110.

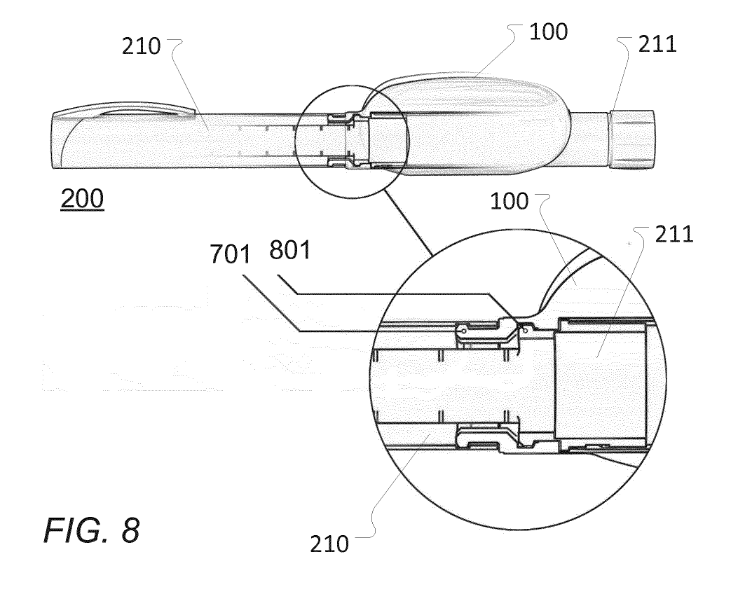

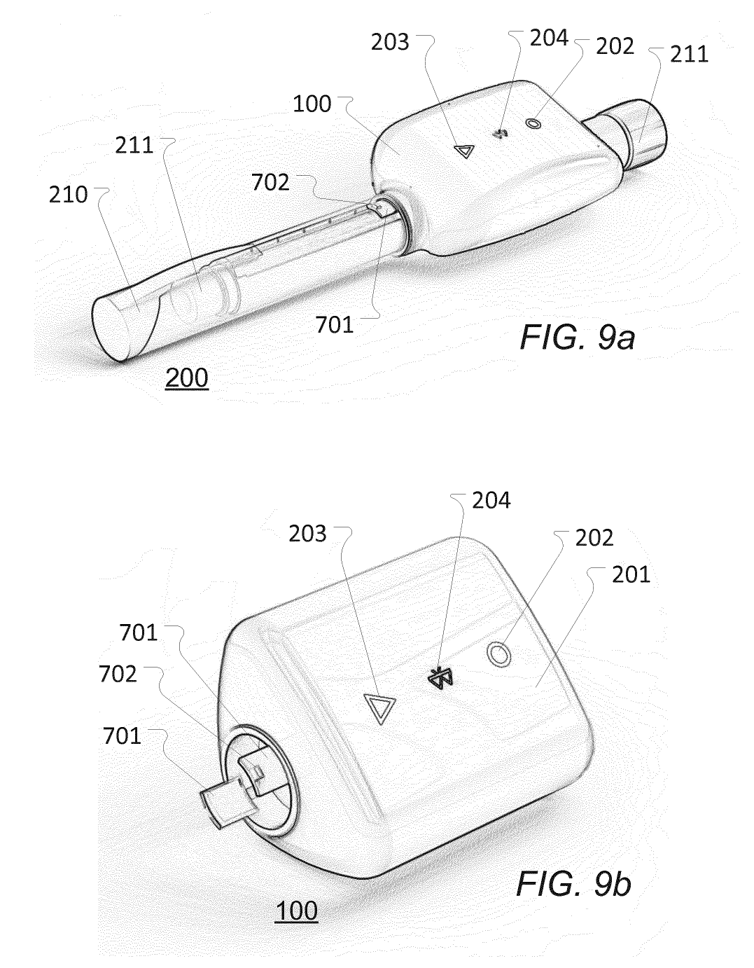

According to a further aspect of the present invention, the position determination device 100 may be attached or secured and (releasably) locked in relation to the drug administration device 200 with the use of a locking mechanism according to various embodiments (see e.g. 701 in FIGS. 8 and 9 for one embodiment) at a predetermined location on and/or predetermined orientation in relation to the drug administration device 200. In this way, it is ensured by such a locking mechanism that a distance from the position determination device 100 to the needle of the drug administration device 200 is always known a priori. This avoids the need for re-calibration every time the position determination device 100 is attached to the drug administration device 200.

In some further embodiments, the position determination device 100 attaches to the drug administration device 200 like a cap of the drug administration device 200 normally would when the position determination device 100 is not present, i.e. the locking mechanism of the position determination device 100 corresponds to the locking mechanism of the cap in that they will be the same or at least similar or compatible. This ensures that the position determination device 100 can be attached or secured to the drug administration device 100 in a reliable way, since the locking mechanism position determination device 100 is a ‘copy’ of the locking mechanism of the cap.

In some further embodiments, the position determination device 100 is further adapted to allow the cap of the drug administration device 200 to be attached to the position determination device 100 rather than directly to the drug administration device 200 itself when the position determination device 100 is attached or secured to the drug administration device 200. This ensures that the cap may still be attached thereby protecting the relevant parts of the drug administration device 200 in a usual way.

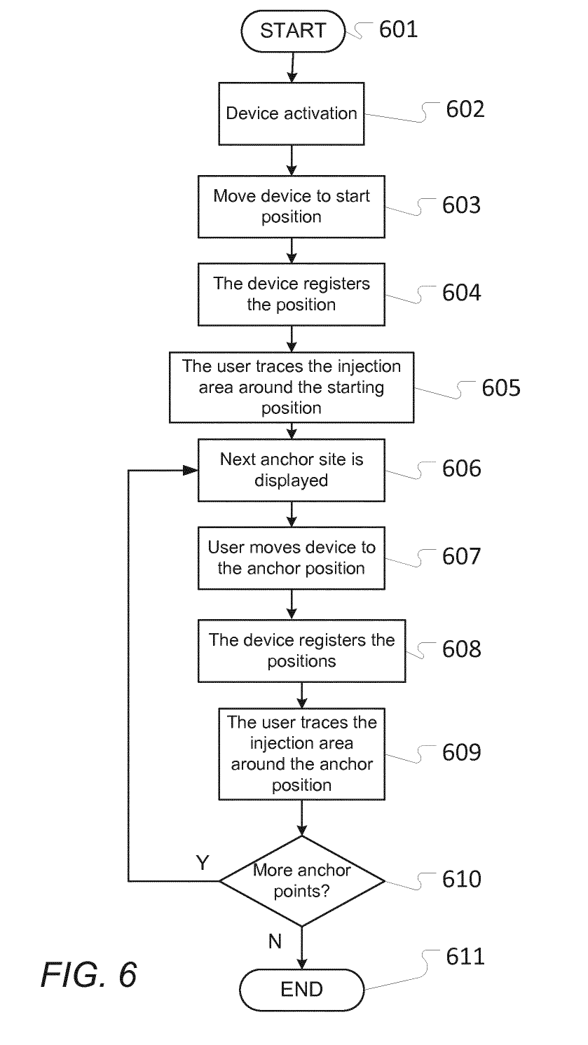

The actual injection location may e.g. be calculated in the following manner: From a known starting position the position determination element 110 is reset to represent a zero-length movement vector. As the position determination device 100 is moved, the position determination element 110 update its internal representation of the distance moved to produce a 3D movement vector. When the position determination device 100 is moved to an anchor point, this movement vector is compared to the calibrated vectors to determine which anchor point the position determination device 100 is at. In addition, data from the position determination element 110 may e.g. be used to determine the relative position in relation to the torso, or the limb where the anchor point is located. The border of the injection area around the anchor points is calibrated e.g. as described in connection with FIG. 6. Using this calibrated area, then the anchor position, the starting position, the exact position of the injection site may be determined when the position determination element 100 (and e.g. the fluid drug administration device 200 as attached or integrated) is moved by the user to a final injection site.

In this way, specific data is obtained of where (and when) one or more drug injections actually have been made. This enables improved injection location handling and/or self-management of the drug administration including e.g. using knowledge of previous injections locations to recommend the next appropriate injection location taking this knowledge into account—e.g. to adhere to and comply with a rotation scheme, a regimen, schedules for administering the drug, etc. and to improve drug absorption and avoid the drawbacks mentioned earlier, to actively warn a user if the user is about to use an injection location that is too close to an earlier injection location (e.g.

within a given time-frame), and further on, as will be explained further in the following.

In some embodiments, the position determination device 100 may automatically transmit, using the wireless communications unit 120, the derived data representing an actual injection location to the separate electric device 300 and/or the fluid drug administration device 200. In this way, the data relating to actual injection is readily made available and may be used for various purposes e.g. as disclosed herein.

In some embodiments, the data representing an actual injection location is derived in response to the user administering the fluid drug using the fluid drug administration device 200. This may in some embodiments be an actual action of the user activating the fluid drug administration device 200 to administer the fluid drug that triggers deriving the data representing an actual injection location.

In some embodiments, the position determination device 100 may also comprise at least one indicator element (see e.g. 202, 203, and 204 in FIGS. 2a-3b and 8-9).

The at least one indicator element (e.g. light, LED, sound emitter, etc.) may be adapted to signal to the user, during use, that a current location of the position determination device 100, as determined by the position determination element 110, is at a location where it is acceptable or advisable to perform an injection taking into account at least the data representing at least one previous injection location.

The at least one indicator element may alternatively or in addition be adapted to signal to the user, during use, that the current location of the position determination device 100, as determined by the position determination element 110, is at a location where it is not acceptable or advisable to perform an injection taking into account at least the data representing at least one previous injection location.

In addition or as an alternative, the separate electric device 300 and/or the fluid drug administration device 200 may comprise at least indicator element performing the same function(s).

One or more of the at least one indicator elements may also be graphical elements as part of a graphical user interface.

The position determination element 110 may e.g. comprise at least one gyroscope and/or at least one accelerometer to determine a location, e.g. in a relative way using fixed known/calibrated positions, as will be explained further in the following. Alternatively, other types of position determination elements may be e.g. including absolute measurement elements.

FIGS. 2a-2d schematically illustrate different views of one exemplary embodiment of a position determination device e.g. as illustrated in FIG. 1.

Shown in FIG. 2a is one exemplary embodiment of a position determination device 100 corresponding, in function at least, to embodiments already described in connection with FIG. 1. This particular embodiment is of a position determination device 100 that is adapted to be attached or secured, during use, to a fluid drug administration device, e.g. like 200 described in connection with FIG. 1 and elsewhere.

The position determination device 100 may e.g. be adapted to be secured to the fluid drug administration device using one or more attachment elements or similar (not shown; see e.g. 701 in FIGS. 8 and 9).

Alternatively, the position determination device 100 may be adapted to be secured to the fluid drug administration device due to its particular shape, e.g. such as shown in the figure and explained further in connection with FIGS. 3a and 3b.

In this and corresponding embodiments, the position determination device 100 comprises one activation or operation button or similar 201 and at least one indicator element 202, 203, 204 such as one or more lights, LEDs, etc. and/or one or more sound emitters used to signal the user of various indications, information, status, etc.

In the shown embodiment, the position determination device 100 comprises a confirmation light or LED 202, a warning light or LED 203, and a wireless communications connection light or LED 204.

The confirmation light or LED 202 may indicate when a given position is ok for drug injection, while the warning light or LED 203 may indicate when a given position is not ok. The connection light or LED 204 may indicate when a wireless connection is established to a separate electric device (e.g. like 300 in FIG. 1).

It should be noted, that the functions of two or more lights or LEDs could be done by fewer lights or LEDs, e.g. only a single one. As an example, one multi-coloured light or LED could be e.g. green for confirmation and red for warning. In addition or as an alternative, the indications could also be audible e.g. using one or more sound emitters.

The indications could also be given on a separate electric device (e.g. like 300 FIG. 1) e.g. in an app or program running on a smartphone, tablet, laptop, PC, etc. instead or in duplication.

Also indicated in FIG. 2a is a proximal and a distal direction. The proximal direction is a direction generally towards the end of the position determination device 100 that normally would point to the injection element of a fluid drug administration device (see e.g. 200 in FIGS. 1 and 3a-3b) that the position determination device 100 is secured to while the distal direction is parallel and opposite to the proximal direction.

It should be noted, that the orientation of the position determination device 100 when secured to the fluid drug administration device in certain embodiments may not be significant. The shown embodiment of the position determination device 100 could e.g. be rotated about its central axis on the fluid drug administration device and it could e.g. also be turned 180° (so the distal end of the position determination device 100 would be in the proximal direction of the fluid drug administration device) while still working.

Shown in FIG. 2b is the position determination device 100 of FIG. 2a shown from ‘above’, i.e. shown from a direction pointing towards the activation or operation button 201 and the indicators 202, 203, and 204.

Shown in FIG. 2c is the position determination device 100 of FIG. 2a shown from one side.

Shown in FIG. 2d is the position determination device 100 of FIG. 2a shown from one end. As can be seen, the position determination device 100 in this view has a more or less circular ring shape with an opening or cut-out 240 for receiving at least a part of the fluid drug administration device when being secured to this.

The position determination device 100 may e.g. have a suitable ‘soft’ or flexible material on its inside on parts that are to be in contact with the fluid drug administration device when attached thereto.

FIG. 3a schematically illustrates a perspective view of the position determination device of FIGS. 2a-2d being secured to a fluid drug administration device.

Shown is the position determination device 100 of FIG. 2a in a state where it is attached or secured to a fluid drug administration device 200 that e.g. may be of the type mentioned in connection with FIG. 1, and in particular a liquid insulin administration device such as an insulin pen or similar.

The fluid drug administration device 200 may be of any suitable type and in this and corresponding embodiments of the position determination device 100 it does not require any modification of the fluid drug administration device to take advantage of the present invention. Moving and using the fluid drug administration device 200 will move the position determination device 100 with a fixed positional relationship between them. If the position determination device 100 is removed and attached back onto the fluid drug administration device 200 at another position there may in certain cases be a need for re-calibration, unless the position and/or orientation of the position determination device 100 on the fluid drug administration device 200 is known apriori as disclosed herein.

FIG. 3b schematically illustrates a perspective view of the position determination device 100 and the fluid drug administration device 200 of FIG. 3a before the position determination device 100 is secured to the fluid drug administration device 200.

In this and corresponding embodiments, the position determination device 100 is simply snapped or clicked onto the fluid drug administration device 200 as indicated by the four broken lines. The shape of the position determination device 100 being generally of a hollow cylindrical shape with its opening or cut-out—as also shown in FIGS. 2a-2d—enables the position determination device 100 to easily but securely be temporarily attached or fixed to the fluid drug administration device 200.

The specific shape of the position determination device 100 of FIG. 3b and corresponding devices are particularly well suited to be secured to pen-type fluid drug administration devices. But the shape may of course vary according to the fluid drug administration device that it is to be used together with.

Alternatively, the position determination device 100 may be ‘slided’ onto the fluid drug administration device 200 from one of the ends. As other alternatives, the position determination device 100 may comprise one or more attachment or securing elements enabling it to be secured to the fluid drug administration device 200 in other ways (whereby the position determination device 100 may have a different shape) e.g. as described in connection with FIGS. 8 and 9.

The fluid drug administration device 200 will typically comprise at least one activation button or other activation elements to initiate the actual administration of the fluid drug to the user.

In some embodiments, the position determination device 100 will derive data representing an actual injection location based on the user specifically activating the fluid drug administration device 200 to administer the fluid drug.

In this way, only one activation action from the user is needed even if the position determination device 100 and the fluid drug administration device 200 are separate devices.

If the position determination device 100 is integrated with the fluid drug administration device 200 this is of course relatively easily enabled.

Even if the position determination device 100 is not integrated with the fluid drug administration device 200 an alternative measure may e.g. be that pressing the activation button or the like on the fluid drug administration device 200 physically will activate the activation button of the position determination device at the same time. It may also be the other way around where activation of the activation button of the position determination device physically will activate an activation button or the like on the fluid drug administration device 200.

This could e.g. be achieved if the position determination device 100 is located on the fluid drug administration device 200 so that the position determination device's activation button is on top of (or in any other engaging relationship) the activation button of the drug administration device 200. It is to be understood that the specific location of the various activation buttons may be different in specific implementations.

Alternatively, a pressure sensor may e.g. be connected to the activation button of the fluid drug administration device 200 to allow the position determination device 100 to register the position at the exact moment of the injection. This pressure sensor may be manual, mechanical (attached to or parallel to the 200 activation mechanism) or may be implemented using a remote distance measurement (like an IR light source and a camera or otherwise). This distance measurement would be used to detect movement of the activation button of the fluid drug administration device 200.

A microphone or other sound sensor may also be used as another alternative for those drug administration devices 200 that incorporates distinct sounds for increase, decrease, and release of the drug dosage. This is common among insulin administration devices to allow users with impaired sight to hear when the dosage is respectively increased, decreased, or released.

In some embodiments (including the ones explained in connection with FIGS. 1 to 2d), the position determination device 100 may further comprises an image sensor or other sensor element adapted to detect an amount of fluid drug being administered by the fluid drug administration device 200. In this way, dosage data may be collected (and communicated further on) by the position determination device 100 in situations where a fluid drug administration device 200 otherwise does not support this.

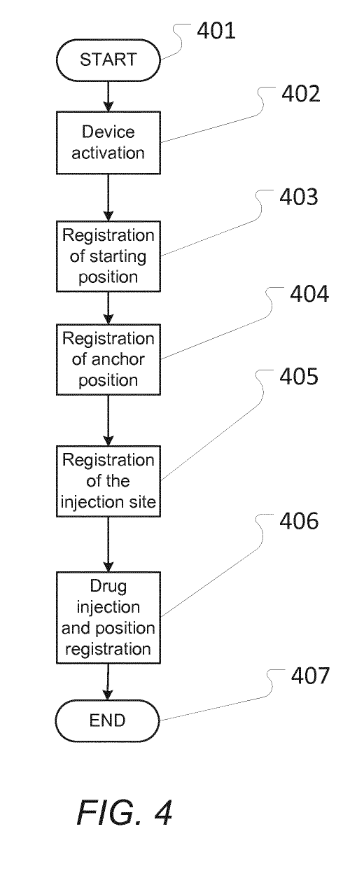

FIG. 4 schematically illustrates a flow chart of one embodiment of position determination by a position determination device as shown in FIGS. 1 to 3b and embodiments thereof.

The method starts or initiates at step 401 and at step 402 the user activates the position determination device (e.g. the position determination device 100 shown and explained in connection with FIGS. 1-3b and variations thereof) by pressing an appropriate activation element (e.g. like 201 shown in FIGS. 2a-3b).

At step 403, the user moves the position determination device, or rather moves the fluid drug administration device (e.g. like 200 shown and explained in connection with FIGS. 1 and 3a-3b) with the attached or integrated position determination device to a given pre-determined or pre-selected starting position. The starting position may e.g. be the navel, a knee, an elbow or any other appropriate starting position as calibrated and may be used as the starting point of any location determination for the associated drug injection. The starting position may e.g. be selected by the user.

After a few seconds of non-movement (or by the user activating an appropriate activation element or some other action or means), the current position (being the starting position) is automatically registered and stored as data in the memory and/or storage of the position determination device.

For relevant embodiments, at least one indicator element, e.g. a confirmation light or LED like 202 in FIGS. 2a-3b, is activated until the position determination device 100 (and the fluid drug administration device) is moved again.

At step 404, the position determination device (and the fluid drug administration device) is moved to another location referred to as an anchor point or the like related to a desired injection location. An anchor point may e.g. be the left knee of the user if the desired injection location is at the left thigh, etc.

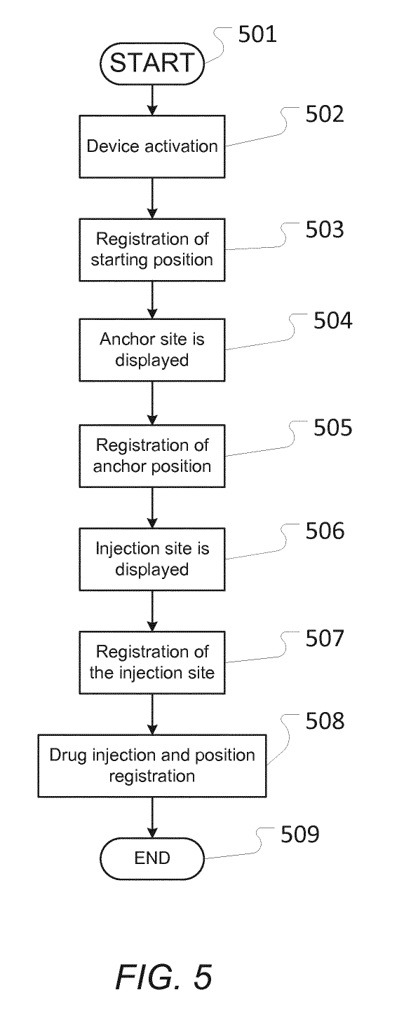

The anchor point location may e.g. be chosen by the user or suggested or recommended e.g. as described in connection with step 504 of FIG. 5.

After a few seconds of non-movement (or by the user activating an appropriate activation element), the current position (being the anchor point) is automatically registered and stored as data in the memory and/or storage of the position determination device.

For relevant embodiments, the least one indicator element, e.g. the confirmation light or LED, is activated until the position determination device (and the fluid drug administration device) is moved again.

In some embodiments, the anchor point is not necessarily needed and step 403 proceeds directly to step 405. This depends on the used position determination scheme.

At step 405, the position determination device (and the fluid drug administration device) is moved to a desired injection location. The desired injection location may e.g. be chosen by the user or suggested or recommended e.g. as described in connection with step 506 of FIG. 5.

If the position of the desired injection location is determined to be too close to previous injection locations (e.g. within about 1 or about 3 cm as advised in the current state of the diabetes treatment art within a given time-frame) by the one or more processing units of the position determination device according to overall general or the user's specifically entered guidelines, then at least one indicator element, e.g. a warning light or LED like 203 in FIGS. 2a-3b, will activate prompting the user that current position is not suitable or recommended for injection and that another injection location should be chosen.

The position determination device (and the fluid drug administration device) should then be moved to a new injection location and when that is done and the new injection location is determined—by the one or more processing units like described above—to be suitable then least one indicator element, e.g. a confirmation light or LED like 202 in FIGS. 2a-3b, is activated to signal that it is ok to perform an injection at the current injection location.

At step 406, the user activates the fluid drug administration device to administer the fluid drug at the injection location and also presses an appropriate activation element (e.g. 201 shown in FIGS. 2a-3b) to register and store the position of the actual injection location as data in the memory and/or storage of the position determination device. As noted previously, it may alternatively be the activation of the fluid drug administration device that triggers the registration and storage of the actual injection location.

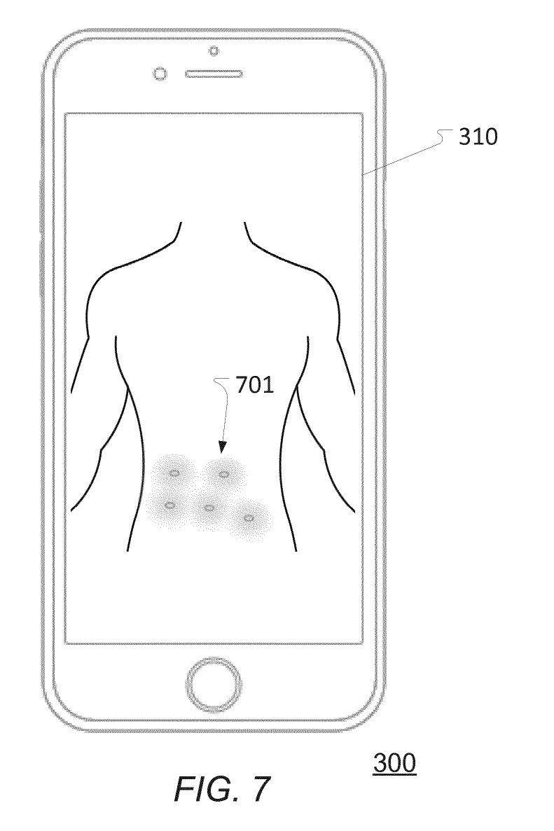

Data representing the position of the actual injection location is then transmitted to a separate electric device (e.g. 300 in FIGS. 1 and 7) and stored there for later use and/or retrieval. After the transmission is completed the position determination device deactivates and the method ends at step 407.

Data representing a number of actual positions may in this way be collected, e.g. together with additional information, time-stamp, amount of administered liquid drug (for embodiments supporting that), etc. This provides a very comprehensive ‘diary’ for the user, which may be useful for a user in improving self-administration routines as well as for medical professionals e.g. to check for regimen compliance, gather data for research, improve patient education, etc.

Using both a start point and an anchor point increases the precision of the registration of the injection location. However, in in some alternative embodiments, an anchor point may be selected in some way (e.g. by the user choosing one or by recommending one) and the injection location is determined relative only to the selected anchor point, i.e. a starting point is not used.

FIG. 5 schematically illustrates a flow chart of one embodiment of a method of suggesting an injection location.

The method starts or initiates at step 501 and at step 502 (which may correspond to step 402 in FIG. 4) the user activates the position determination device (e.g. the position determination device 100 shown and explained in connection with FIGS. 1-3b and variations thereof) by pressing an appropriate activation element (e.g. 201 shown in FIGS. 2a-3b).

At step 503 (which may correspond to step 403 in FIG. 4), the user moves the position determination device and the fluid drug administration device (e.g. 200 shown and explained in connection with FIGS. 1 and 3a-3b) to a given pre-determined or pre-selected starting position, e.g. like the navel, a knee, an elbow, or any other appropriate starting position as calibrated.

After a few seconds of non-movement (or by some other action or means), the current position (being the starting position) is automatically registered and stored as data in the memory and/or storage of the position determination device. For relevant embodiments, at least one indicator element, e.g. a confirmation light or LED like 202 in FIGS. 2a-3b, is activated until the position determination device 100 (and the fluid drug administration device) is moved again.

At step 504, an appropriate injection location to use is derived.

The injection location to use this time may be derived by a separate electric device (e.g. 300 in FIGS. 1 and 7). Alternatively, it may be derived by the one or more processing units of the position determination device or a fluid drug administration unit.

The inject location to use next is derived according to general or the user's specifically entered guidelines. This may for example be by a scheme where the determination of injection sites follow a circular or spiral pattern across the belly to ensure an even spread of sites over a given period like a week, for example a linear upward moving of sites on the thighs alternating between the right and the left, or a time-based guideline always advising about the injection site unused for the longest amount of time to ensure the largest spread. The guidelines used should be chosen based on dialogue with medical professionals and individual preferences.

After the next injection location to use has been derived, an anchor point associated with the given next injection location is then presented to the user, e.g. on a display of the separate electric device or in another suitable way.

At step 505, the user moves the fluid drug administration device (and thereby the position determination device) to the presented anchor point or the like of the suggest injection location. If the user moves the fluid administration device and the position determination device to a different location than the suggested position, a warning may e.g. displayed to the user (e.g. using at least one indicator element such as a warning light or LED like 203 in FIGS. 2a-3b) and/or on a separate electric device (e.g. 300 in FIGS. 1 and 7).

After a few seconds of non-movement (or by some other action or means), the current position (being the anchor point) is automatically registered and stored as data in the memory and/or storage of the position determination device. For relevant embodiments, the least one indicator element, e.g. the confirmation light or LED, is activated until the position determination device (and the fluid drug administration device) is moved again.

In some embodiments, the anchor point is not necessarily needed and step 503 proceeds directly to step 506. This depends on the used position determination scheme

At step 506, the injection location (as being associated with the registered anchor point) to use next as derived at step 504 is displayed e.g. on the display of the separate electric device or in another suitable way.

At step 507, the user moves the fluid drug administration device (and thereby the position determination device) to the presented suggested injection location.

For relevant embodiments, at least one indicator element, e.g. a warning light or LED like 203 in FIGS. 2a-3b, will activate and stay activated until the position determination device is moved within a predetermined threshold distance of the injection location that was displayed at step 506.

The predetermined threshold may be defined in the user's entered guidelines or be an overall general threshold.

For relevant embodiments, when the position determination device comes within the predetermined threshold distance of the injection location then least one indicator element, e.g. a confirmation light or LED like 202 in

FIGS. 2a-3b, is activated to signal that it is ok to perform an injection at the current injection location.

At step 508, the user activates the fluid drug administration device to administer the fluid drug at the injection location and also presses an appropriate activation element (e.g. 201 shown in FIGS. 2a-3b) to register and store the position of the actual injection location as data in the memory and/or storage of the position determination device. As noted previously, it may alternatively be the activation of the fluid drug administration device that triggers the registration and storage of the actual injection location.

Data representing the position of the actual injection location is then transmitted to the separate electric device (e.g. 300 in FIGS. 1 and 7) and stored there for later use and/or retrieval. After the transmission is completed the position determination device deactivates and the method ends at step 509.

FIG. 6 schematically illustrates a flow chart of one embodiment of a calibration method of a position determination device.

The method starts or initiates at step 601 and at step 602 the user activates the position determination device (e.g. the position determination device 100 shown and explained in connection with FIGS. 1-3b and variations thereof) by pressing an appropriate activation element (e.g. 201 shown in

FIGS. 2a-3b).

At step 603, the user moves the position determination device, or rather moves the fluid drug administration device (e.g. 200 shown and explained in connection with FIGS. 1 and 3a-3b) with the attached or integrated position determination device to a given pre-determined or pre-selected starting position and keeps the position determination device still. It may be that the user chooses the starting position him- or herself or that it is pre-defined and presented in some way, e.g. using a separate electric device (e.g. 300 in FIGS. 1 and 7).

This starting position is intended to be used for the following position determinations (e.g. at least until a next (re-)calibration.

At step 604, the user activates an appropriate activation element and the current position (being the starting position) is then registered and stored as data in the memory and/or storage of the position determination device.

At step, 605 the user indicates the boundary of the injection area that is to be associated with the starting position (if there is one to be associated). In some embodiments, this may be done by the user moving the position determination device from the starting position to an edge of the injection area and then while keeping the activation element active or pressed, the user traces the edge of the injection area with the position determination device (being attached or integrated with the fluid drug administration device). Alternatively, other ways to indicate the boundary and/or the injection area may be used.

The positon determination device then registers and stores the injection area and/or boundary as data in its memory and/or storage and transmits the data to the separate electric device (e.g. 300 in FIG. 1) for storage and/or visual feedback to the user.

At step 606, the separate electric device displays information to the user about which anchor position to register.

At step 607, the user moves the position determination device and the fluid drug administration device to the shown anchor position.

At step 608, the current position (being the anchor point) is automatically registered and stored as data in the memory and/or storage of the position determination device e.g. by keeping the position determination device still for a predetermined amount or time or by activating an appropriate activation element (e.g. 201 shown in FIGS. 2a-3b). For relevant embodiments, the least one indicator element, e.g. the confirmation light or LED, is activated until the position determination device (and the fluid drug administration device) is moved again.

At step 609, the user indicates the boundary of the injection area that is to be associated with the current anchor position and it is registered and stored in the same manner as at step 605 for the starting position.

At step 610 it is checked whether this procedure is to be done for any further anchor points. If yes, the method loops back to step 606 whereby steps 606-610 is repeated in connection with the next anchor point. If no (signifying no remaining anchor points), the calibration process terminates at step 611.

In this way, simple and reliable calibration is provided.

FIG. 7 schematically illustrates a separate electric device displaying a ‘heatmap’ view or similar of earlier injections.

Shown is a separate electric device 300, e.g. corresponding to the one shown in FIG. 1, having a display 310 to be used according to some aspects in connection with some embodiments of a position determination device (such as 100 shown in FIGS. 1-3b, 8-9, and variations thereof) and a fluid drug administration device (such as 200 shown in FIGS. 1 and 3a-3b, 8-9, and variations thereof).

As already mentioned, actual injections locations may preferably be registered on an ongoing basis using the position determination device and the fluid drug administration device.

According to these aspects, the actual injection locations may be shown as a graphical representation of data generally known as a ‘heat map’ or similar where the individual values (location e.g. together with further relevant information like time of injection, actual amount of injected fluid drug, etc.) are represented as colors in a color scheme superimposed on a picture or a graphic representation of the overall injection area(s), e.g. like the torso, the thighs, upper arms, or what other injection area the user has defined, etc. The values may e.g. be stored in a matrix data structure and/or any other suitable data structure.

The color scheme may e.g. be one from green to red where a given injection location is marked yellow if it was taken in (too) close proximity (e.g. within 1 or 3 cm, or as chosen by the user) to one another injection location made within a predetermined time (e.g. a week or other timeframe as e.g.

determined by the user), and red if it is in (too) close proximity to 2 or more injection locations, and green in all other cases.

The heatmap may be updated from the device when activated thus enabling the user to check an intended injection location in relation to previous injection locations.

In a recommendation mode (e.g. as indicated in FIG. 5), the device itself may show status of injection locations based om the same color scheme or recommend locations based on the chosen rotation principles.

The aspect(s) of showing previous injection locations like this may be used independently of the described aspects and embodiments of the position determination device although they together work especially well.

FIG. 8 schematically illustrates an embodiment of a locking mechanism for attaching an embodiment of a position determination device to a drug administration device.

Shown are an embodiment of a position determination device 100 as disclosed herein being attached to a drug administration device, here in the form of a insulin pen or other pen type fluid drug administration device, as well as an enlargement illustrating further details.

In this and corresponding embodiments, the position determination device 100 as disclosed herein comprises one exemplary embodiment of an attachment element in the form of a locking mechanism 701 adapted to releasably engage with a mating or corresponding locking mechanism 801 of the liquid drug administration device 200. The shown drug administration device 200 comprises a cap 210 and a main body 211. More specifically, the locking mechanism 701 is adapted to engage with a locking mechanism 801 of the main body 211 of the drug administration device 200 that normally is used to engage a locking mechanism of the cap 210, i.e. the locking mechanism 701 of the position determination device 100 corresponds to the locking mechanism of the cap in that they will be the same or at least similar or compatible.

In some further embodiments, and as shown, the locking mechanism 701 is further adapted to engage with the locking mechanism of the cap 210 (see e.g. 702 in FIG. 9) so that the cap 210 still can be attached to the drug administration device 200 even with the position determination device 100 in place, now just connecting with the position determination device 100 instead of directly to the drug administration device 200.

The locking mechanism 701 allows the user an easy way to attach/detach the position determination device 100 to/from the drug administration device 200 in a reliable way. The specific embodiment of such a locking mechanism 701 will typically depend on the specific shape, size, and form of the cap 210 of the drug administration device 200 and how this attaches to the main body of the drug administration device 200.

FIGS. 9a and 9b schematically illustrate an embodiment of the position determination device attached to a drug administration device and by itself.

Shown in FIG. 9a is illustrated an embodiment of the position determination device 100 attached to a drug administration device 200 as disclosed herein where the cap 210 of the drug administration device 200 is also attached to the main body 211 of the drug administration device 200. The cap 210 is shown transparently or with a cut-out to illustrate parts of the main body 211 of the drug administration device 200. The attachment is done using a locking mechanism 701 as disclosed herein. In some embodiments of the locking mechanism 701 and as shown it comprises an engaging element 702 for allowing a locking mechanism of the cap 210 to engage so that the cap 210 may be attached to the position determination device 100 instead of to the main body 211 of the drug administration device 200.

The shown drug administration device 200 also comprises an activation button 201, as well as a number of indicator elements here in the form of a confirmation light or LED 202, a warning light or LED 203, and a wireless communications connection light or LED 204 for user feedback as disclosed herein.

In some embodiments as disclosed herein, the position determination device further comprises a temperature sensor adapted to continuously monitor whether the liquid drug is kept at safe temperature. A warning may be given before the temperature reaches levels that will make the liquid drug unfit for use. This warning may be given using audiovisual cues and/or by the separate electric device.

According to an alternate aspect of the invention, image recognition may be used as a method of locating injection sites.

Even though, the description mainly mention user-administrated injection where a person to receive the liquid drug is also the user to administer the drug it is to be understood that at least for certain aspects, the user may e.g. be a medical professional or the like.

Some of the different components are only disclosed in relation to a single embodiment of the invention, but is meant to be included in the other embodiments without further explanation.

Some preferred embodiments have been shown in the foregoing, but it should be stressed that the invention is not limited to these, but may be embodied in other ways within the subject matter defined in the following claims.

In the claims enumerating several features, some or all of these features may be embodied by one and the same element, component or item. The mere fact that certain measures are recited in mutually different dependent claims or described in different embodiments does not indicate that a combination of these measures cannot be used to advantage.

It should be emphasized that the term “comprises/comprising” when used in this specification is taken to specify the presence of stated features, elements, steps or components but does not preclude the presence or addition of one or more other features, elements, steps, components or groups thereof.

1. A position determination device for determining and registering an injection location of administered fluid drug administration on a user, wherein the position determination device comprises
a position determination element configured to determine a current location of the position determination device, and
one or more processing units configured to derive data representing an actual injection location based on a current location determined by the position determination unit.

2. The position determination device according to claim 1, wherein the one or more processing units are configured to derive the data representing an actual injection location based on a current location determined by the position determination unit in response to the user administering the fluid drug using a fluid drug administration device.

3. The position determination device according to claim 1, wherein
the position determination device is further configured to be secured to the fluid drug administration device.

4. The position determination device according to claim 3, wherein the position determination device has a generally hollow cylindrical shape with an opening or cut-out configured to receive at least a part of the fluid drug administration device when being secured to this.

5. The position determination device according to claim 3, wherein the position determination device comprises a locking mechanism adapted to releasably engage with a mating or corresponding locking mechanism of the liquid drug administration device thereby attaching the position determination device at a predetermined location on and/or predetermined orientation in relation to the drug administration device.

6. The position determination device according to claim 5, wherein the locking mechanism corresponds to a locking mechanism of a cap of the drug administration device.

7. The position determination device according to claim 5, wherein the locking mechanism is further adapted to releasably engage with a mating or corresponding locking mechanism of a cap of the liquid drug administration device thereby enabling the cap to be attached to the position determination device.

8. The position determination device according to claim 1, wherein
the position determination device further comprises a wireless communications unit configured to automatically transmit the derived data representing an actual injection location to a separate electric device.

9. The position determination device according to claim 1, wherein
an actual action of the user activating the fluid drug administration device to administer the fluid drug triggers deriving the data representing an actual injection location.

10. The position determination device according to claim 9, wherein a mechanical or remote sensor is attached directly to or attached in connection with an activation button of the fluid drug administration device where the mechanical or remote sensor is configured to detect movement of the activation button of the fluid drug administration device.

11. The position determination device according to claims 9, wherein the position determination device further comprises a sound sensor configured to register one or more distinct sounds caused by a user operating the drug administration device to increase, decrease, and/or release a drug dosage as an actual action of the user and wherein the position determination device is adapted to trigger deriving the data representing an actual injection location in response to registering the one or more distinct sounds.

12. The position determination device according to claim 1, wherein the position determination device further comprises
a memory and/or storage comprising data representing at least one previous injection location,
at least one indicator element configured to signal to the user
that the current location of the position determination device, as determined by the position determination element, is at a location where it is acceptable or advisable to perform an injection taking into account at least the data representing at least one previous injection location, and/or
that the current location of the position determination device, as determined by the position determination element, is at a location where it is not acceptable or advisable to perform an injection taking into account at least the data representing at least one previous injection location.

13. The position determination device according to claim 1, wherein the position determination element comprises at least one gyroscope or at least one accelerometer.

14. The position determination device according to claim 1, wherein the position determination device is further configured to register an actual amount of administered liquid drug and/or a time-stamp when deriving data representing an actual injection location.

15. The position determination device according to claim 1, wherein the position determination device further comprises an image sensor or other sensor element adapted to detect an amount of fluid drug being administered by the fluid drug administration device.

16. The position determination device according to claim 1, wherein the position determination device is further configured to
register a starting position in response to the user moving the position determination device to a pre-determined starting position,
register a location of an anchor position in response to the position determination unit determining the movement from the starting position to the anchor position,
register a location of the actual injection location in response to the position determination unit determining the movement from the anchor position to the injection location.

17. The position determination device according to claim 16, wherein the position determination device is further configured to
recommend the anchor position and/or recommend the location of the actual injection location in response to a rotation schema taking into account one or more previously registered actual injection locations.

18. The position determination device according to claim 1, wherein the position determination device is further configured to
register a starting position when the user has moved the position determination device to a starting position,
if used, registering a region in connection with the starting position defining a region suitable for receiving injections,
registering one or more anchor points, by determining their relative position in relation to the starting position, and for each anchor point registering a region defining a region associated with the given anchor point being suitable for receiving injections.

19. The position determination device according to claim 1, wherein the fluid drug is one selected from the group consisting of: insulin, drugs for multiple sclerosis therapies with interferons and immune suppressives, drugs for hormone therapies, drugs for growth hormone therapies, and drugs for anti-/hormone followup treatments to breast or prostate cancer.

20. The position determination device according to claim 1, wherein the position determination device is integrated with a fluid drug administration device and wherein the derived data representing an actual injection location is made available to at least another electric element of the fluid drug administration device.

21. The position determination device according to claim 1, wherein the position determination device is for determining and registering an injection location of self-administered fluid drug administration on a user, or the position determination device is for determining and registering an injection location of administered fluid drug administration on a user administered by a medical professional.

22. Use of a position determination device according to claim 1, to register one or more locations of actual injection locations.
